(19) United States
(12) Patent Application Publication
(10) Pub. No.: US 2013/0078705 A1
(43) Pub. Date: Mar. 28, 2013

(54) PROTEASE FOR TREATING NAIL ABNORMALITIES

(71) Applicant: Hsuehkuan LU, Taipei City (TW)

(72) Inventor: Hsuehkuan LU, Taipei City (TW)

(21) Appl. No.: 13/241,097

(22) Filed: Sep. 22, 2011

(51) Int. Cl. C12N 9/76; C12N 9/66; C12N 9/54; C12N 9/50

(52) U.S. Cl. 435/213; 435/219; 435/218; 435/221

(57) ABSTRACT

A protease for treating nail abnormalities is a type of protease. Since nails are mostly composed of keratin protein, the optimum protease for decomposing the main component (keratin) of the nail is keratinase or serine protease which is functionally similar to keratinase. The serine protease include trypsin, chymotrypsin, elastase, subtilisin, etc, these enzymes can be used to treat pathologically or mechanically damaged nail tissue to thin or remove keratin which is the key component of the nail, thus facilitating drug delivery in later treatment.

### BACKGROUND OF THE INVENTION

1. Field of the Invention

The present invention relates to the protease for treating nail abnormalities, especially Tinea unguium. The optimum enzymes for decomposing the main component (keratin) of the nail are keratinase or serine protease which is functionally similar to keratinase.

2. Description of the Prior Art

Onychomycosis is also called Tinea unguium. Statistically, onychomycosis has a positive correlation with diabetes, circulatory disease, immunodeficiency disease, nail contusion and humid environment. Epidemiological surveys have shown that the prevalence of onychomycosis is approximately 30%.

There are many groups of fungi cause onychomycosis, but 85-90% of these diseases are caused by the dermatophyte group of fungi. Toenails are four times more commonly affected by the infectious organism of onychomycosis than fingernails. Dermatophyte often leads to superficial white onychomycosis and cause white brickle area in the fingernails, however, non-dermatophyte moulds can also cause such changes, and 5% of cases are due to non-dermatophyte moulds. Mixed infection by dermatophyte group of fungi and non-dermatophyte moulds can account for 5% of the total onychomycosis nail. All onychomycosis caused by whatever reasons might cause total dystrophic onychomycosis, the nails become thick, yellow, brickle and malformed.

Onychomycosis is not a fatal disease but would cause uncomfortable feeling. Patients with onychomycosis nail are often embarrassed by strange looks from other people when the affected part gets exposed. Onychomycosis nail was thought to be difficult to be cured in the past. Curative effect of the incomplete treatment is always not good. Once pathogenic organisms grow in the deep of nail plate and nail bed, treatment would be very difficult and recurrence rate is high. With increasing contacts among people, the number of cases of onychomycosis also has a tendency to increase. In recent years, some new oral medicines can perform a relatively good curative effect, such as grisoefulvin, terbinafine, (Lamisil) and itraconazole (Sporanox), etc, the curative rate of which is approximately up to 35˜50% in a short treatment time (six weeks for fingernail infection and 12 weeks for toenail infection), however, the side effects often cause treatment interruption, and these side effects include nausea, headache, upset stomach, photaesthesia, etc, and even fatal cases caused by fulminant hepatitis. The patients on oral antifungal drugs have to have their liver function tested every 4-6 weeks. External medicine for treating onychomycosis has few side effects, but the curative effect is not good, because nails are mostly composed of keratin protein to protect fingers or toes and are waterproof, external drugs, such as creams or drops, are unable or difficult to penetrate the nail and reach the focus.

To improve the curative effect of the external drugs on onychomycosis, the existing method is to remove the whole or part of the nail by operation. However, this method has the side effects of pain, discomfort or even bad looking of the nails or nail beds, especially for female patients or for those who care much about their appearance, removing nails is not a good choice.

To solve the abovementioned disadvantages, the author of this application, after lots of trials and tests, has finally developed enzymes for treating nail abnormalities, especially Tinea unguium, on the premise of taking into consideration of practicality, curative effect and beauty of the nails.

### SUMMARY OF THE INVENTION

The primary object of the present invention is to provide enzymes capable of treating the nail of Tinea unguium. Prior to treatment of the nail of Tinea unguium, keratinase or serine protease which is functionally similar to the keratinase is applied to thin or remove the keratin—the key component of the nail. Hence, it is easier for the follow-up drugs for treating the nail to reach the target tissue, consequently facilitating the drugs to exert their therapeutic effect. Moreover, for the nail abnormalities caused by other reasons or for physical damage to the nail, it can also use the enzyme to thin or completely remove the keratin which is the key component of the nail. This is good for the beauty of the nail which will grow back in the future.

The main component of nails is protein, and mostly keratin. Keratinase and serine protease are substrate specific and therefore are used to decompose the key component of nail.

### DETAILED DESCRIPTION OF THE PREFERRED EMBODIMENTS

The present invention will be clearer from the following description, the preferred embodiment in accordance with the present invention.

Tinea pedis is a disease of skin fungal infection, which will cause the feeling of itchy, burning and skin peel. The various funguses causing tinea pedis may spread to other parts of the body, such as nail bed and groin. The nail fungal infection is called tinea unguium, which is commonly found throughout the world. A statistic from National Institute of Health of America shows that 2-13% of the North America people are infected with tinea unguium, and at least 15-20% of the people aged 40-60 have one or more infected toenails or fingernails, ranging from the light infection which only causes the color change of the nail to the severe infection which causes nail separation and even malformation of the fingernails and toenails.

Topical drugs for treating tinea unguium can only be delivered to the target focus (the infected bed nail) by diffusing or permeating through the nail. However, nails are more like hair than corneum in terms of chemical composition and permeability, and nitrogen is the main component of the nails, which reflects the protein property of the nails. Nails are 100-200 times thicker than corneum and have the characterization of non-permeable, hence, almost no drugs can permeate through the nails to reach the focus. In other words, to improve the curative effect and make the drugs reach the focus, it has to remove or thin the nail prior to medical treatment.

The method for removing or thinning nails in accordance with the present invention is the use of keratinase (a kind of enzyme) or serine protease which is specific to pathologically or mechanically damaged nail tissue to thin or remove the keratin which is the key component of the nail, making it easier for the follow-up drugs for treating the nail to reach the target tissue, consequently facilitating the drugs to exert their therapeutic effect. Moreover, for the nail abnormalities caused by other reasons or for physical damage to the nail, it can also use the enzyme to thin or completely remove the keratin which is the key component of the nail. This is good for the beauty of the nail which will grow back in the future.

Keratinase existing in nature (formed by nature) generally comes from the fungi or bacteria for causing the Tinea unguium. When commercially produced (artificial) keratinase is applied to a people, the people may feel the same as the patient with Tinea unguium. In other words, Tina unguium is a life-long and chronic infection. The nails and peripheral tissues of the patient with Tina unguium will be affected by the keratinase excreted from the fungi or bacteria for causing the Tina unguium. When the commercially produced keratinas in accordance with the present invention is applied to a people, the people won't feel the side effect (such as pain or anything) just like the patient with tinea unguium. Furthermore, the side effect can be controlled to the safety or acceptable level by controlling the dosage or treatment time, even if there is side effect.

The optimum protease for decomposing the main component (keratin) of the nail is keratinase or serine protease which is functionally similar to keratinase. The serine protease include trypsin, chymotrypsin, elastase, subtilisin, etc, and the present invention includes but is not limited to the above listed serine protease. The protease can be single component, compound components or one of compound components, or can be one of the compound components of other nail or tinea unguium nail drugs, which can be applied to nail by spraying, taping or any other means and has a pH ranging from 1-14, and preferably more than 7 (alkaline). Furthermore, the treatment temperature is not limited, normal, high or low temperature are all ok. The efficiency of the keratinase or serine protease treating (decomposing) the nail would be improved when the keratinase or serine protease is added with denatuaration of protein, such as urea or guanidine. Since enzyme is composed of protein, and the protease of the present invention will definitely decompose protease itself and will gradually lose its effectiveness during treatment.

To summarize, the enzyme (protease) in accordance with the present invention can be used to pre-treat the nail (to thin or remove the nail) prior to treatment of the nail of Tinea unguium, making it easier for the follow-up drugs for treating the nail to reach the target tissue, consequently facilitating the drugs to exert their therapeutic effect. Moreover, it could have better appearance of the nail when the enzyme treated nail regenerate in the future.

While we have shown and described various embodiments in accordance with the present invention, it is clear to those skilled in the art that further embodiments may be made without departing from the scope of the present invention.

What is claimed is:

1. An enzyme for treating nail abnormalities being protease and used to treat pathologically or mechanically damaged nail tissue to thin or remove keratin which is the key component of the nail, thus facilitating drug delivery in later treatment.

2. The enzyme for treating nail abnormalities as claimed in claim 1, wherein the protease is keratinase.

3. The enzyme for treating nail abnormalities as claimed in claim 1, wherein the protease is serine protease.

4. The enzyme for treating nail abnormalities as claimed in claim 1, wherein the protease is single component.

5. The enzyme for treating nail abnormalities as claimed in claim 1, wherein the protease is compound components or one of compound components.

6. The enzyme for treating nail abnormalities as claimed in claim 1, wherein the protease is one of the compound components of other nail or tinea unguium nail drugs.

7. The enzyme for treating nail abnormalities as claimed in claim 3, wherein the serine protease is trypsin.

8. The enzyme for treating nail abnormalities as claimed in claim 3, wherein the serine protease is chymotrypsin.

9. The enzyme for treating nail abnormalities as claimed in claim 3, wherein the serine protease is elastase.

10. The enzyme for treating nail abnormalities as claimed in claim 3, wherein the serine protease is subtilisin.
